(19) United States
(12) Patent Application Publication
(10) Pub. No.: US 2024/0408052 A1
(43) Pub. Date: Dec. 12, 2024

(54) COMPOSITION CONTAINING SESAMIN AND ERGOTHIONEINE OR SALT THEREOF

(71) Applicant: SUNTORY HOLDINGS LIMITED, Osaka-shi, Osaka (JP)

(72) Inventors: Chie Abe, Soraku-gun (JP); Norifumi Tateishi, Soraku-gun (JP); Akira Matsuura, Chiba-shi (JP)

(73) Assignee: SUNTORY HOLDINGS LIMITED, Osaka-shi, Osaka (JP)

(21) Appl. No.: 18/699,069

(22) PCT Filed: Oct. 7, 2022
(86) PCT No.: PCT/JP2022/037532

§ 371 Date: Apr. 5, 2024

(30) Foreign Application Priority Data
Oct. 13, 2021 (JP) 2021-168087

(51) Int. Cl. A61K 31/36; A61K 31/4172; A61P 43/00

(52) U.S. Cl. CPC A61K 31/36; A61K 31/4172; A61P 43/00

(57) ABSTRACT

The present invention aims to provide a composition capable of effectively suppressing cellular senescence. The present invention relates to a composition containing at least one sesamin-class compound and ergothioneine or a salt thereof, wherein a ratio of a weight of the sesamin-class compound to a weight of ergothioneine or a salt thereof in terms of ergothioneine, i.e., sesamin-class compound/ergothioneine, is 0.05 to 50.

### TECHNICAL FIELD

The present invention relates to a composition containing at least one sesamin-class compound and ergothioneine or a salt thereof. The present invention also relates to, for example, a method of suppressing cellular senescence.

### BACKGROUND ART

Cellular senescence refers to a state of irreversible arrest of cell growth due to genetic damage or the like. For example, normal somatic cells of humans and other mammals have a limit to the number of times the cells can divide. After a certain number of repeated cell divisions, the cells undergo irreversible arrest of cell division.

Unlike apoptosis, the cells undergoing senescence (senescent cells) do not kill themselves. Thus, senescent cells accumulate in tissues and organs with aging (Non-Patent Literature 1). Senescent cells are not only arrested in the cell cycle but also show a decline in cellular function. Physiologically active factors secreted from senescent cells damage surrounding normal cells, causing a decline in organ function or the like. Thus, senescent cells are regarded as the cause of senescence at the individual level. Meanwhile, it has been reported that suppression of senescent cell accumulation extends the lifespan (Non-Patent Literature 2). The above-described nature of senescent cells has brought an increase in interest in suppressing cellular senescence.

A sesamin-class compound is one of main lignan compounds found in sesame and has been reported to have an anti-fatigue action and the like (Patent Literature 1). Ergothioneine is an amino acid found in mushrooms and the like. L-ergothioneine is present in nature. Ergothioneine has been reported to have physiological actions such as an action that promotes immune response activation cytokine production (Patent Literature 2).

### CITATION LIST

### Patent Literature

Patent Literature 1: WO 2008/126587
Patent Literature 2: JP 2017-218431 A

### Non-Patent Literature

Non-Patent Literature 1: Herbig U, Ferreira M, Condel L, Carey D, Sedivy JM. “Cellular Senescence in Aging Primates.” Science. 2006; 311 (5765): 1257-1257.
Non-Patent Literature 2: Baker, Darren J., et al. “Naturally occurring p16 Inkda-positive cells shorten healthy lifespan.” Nature 530. 7589 (2016): 184-189.

### SUMMARY OF INVENTION

### Technical Problem

However, no report has been made on the action of a composition containing a sesamin-class compound and ergothioneine on cellular senescence.

The present invention aims to provide a composition capable of effectively suppressing cellular senescence.

### Solution to Problem

As a result of extensive studies to solve the above problem, the present inventors found that cellular senescence can be effectively suppressed by using at least one sesamin-class compound in combination with ergothioneine or a salt thereof.

The present invention encompasses the following composition and the like.

(1) A composition containing: at least one sesamin-class compound; and ergothioneine or a salt thereof, wherein a ratio of a weight of the sesamin-class compound to a weight of ergothioneine or a salt thereof in terms of ergothioneine, i.e., sesamin-class compound/ergothioneine, is 0.05 to 50.

(2) The composition according to (1) above, wherein the sesamin-class compound is at least one of sesamin or episesamin.

(3) The composition according to (1) or (2) above, wherein the composition is an oral composition.

(4) The composition according to any of (1) to (3) above, wherein the composition is a food or beverage.

(5) The composition according to (1) or (2) above, wherein the composition is a cosmetic.

(6) The composition according to any of (1) to (5) above for use in suppressing cellular senescence.

(7) The composition according to any of (1) to (6) above, wherein the composition is an anti-senescence composition.

(8) The composition according to any of (1) to (7) above, wherein the composition is labeled with at least one selected from the group consisting of “suppressing cellular senescence”, “suppressing conversion into senescent cells”, “preventing cellular senescence”, “preventing senescent cell formation”, “suppressing an increase in senescent cells”, “reducing senescent cell accumulation”, “delaying cellular senescence”, “delaying progression of cellular senescence”, “suppressing cellular senescence induction”, “increasing percentage of young cells”, “extending healthy lifespan”, “healthy longevity”, “young cells”, “youthful as a whole”, and “youthful look”.

(9) A method of suppressing cellular senescence, the method including administering the composition according to any of (1) to (8) above.

(10) Use of the composition according to any of (1) to (8) above for suppressing cellular senescence.

### Advantageous Effects of Invention

The present invention can provide a composition capable of effectively suppressing cellular senescence.

### BRIEF DESCRIPTION OF DRAWINGS

FIG. 1 is a graph showing results of studies on a cellular senescence-suppressing effect of a sesamin-episesamin mixture and ergothioneine.

FIG. 2 is a graph showing results of studies on the cellular senescence-suppressing effect of the sesamin-episesamin mixture and ergothioneine.

FIG. 3 is a graph showing results of studies on a lifespan-extending effect of the sesamin-episesamin mixture and ergothioneine on yeast cells.

### DESCRIPTION OF EMBODIMENTS

The composition of the present invention is a composition containing at least one sesamin-class compound and ergothioneine or a salt thereof, and the ratio of the weight of the sesamin-class compound to the weight of ergothioneine or a salt thereof in terms of ergothioneine, i.e., sesamin-class compound/ergothioneine, is 0.05 to 50.

In the present invention, the term “sesamin-class compound” is a collective term for compounds including sesamin and its analogs. Sesamin is one of main lignan compounds found in sesame. Examples of the sesamin analogs include episesamin and dioxabicyclo[3.3.0]octane derivatives described in JP H04-9331 A. The at least one sesamin-class compound may include one of these compounds alone or two or more of these compounds. Specific examples of the sesamin-class compound include sesamin, episesamin, sesaminol, episesaminol, sesamol, and sesamolin. Stereoisomers or racemates of these compounds may be used alone or in mixture. In the present invention, the at least one sesamin-class compound preferably includes sesamin and/or episesamin, and more preferably includes sesamin and episesamin. When sesamin and episesamin are used, the ratio thereof is not limited, but the weight ratio of sesamin:episesamin is preferably 1:0.1 to 1:9, more preferably 1:0.3 to 1:3, still more preferably 1:0.5 to 1:2.

The sesamin-class compound for use in the present invention is not limited in any way by its form, production method, or the like. For example, when the sesamin-class compound is sesamin, a sesamin-containing extract obtained from sesame oil by a known method (e.g., the method described in JP H04-9331 A) can be used (hereinafter, such a sesamin-containing extract is referred to as a “sesamin extract” or “purified sesamin”). Commercially available sesame oil (in liquid form) can also be used as is. However, when sesame oil is used, the characteristic flavor of sesame oil is sometimes evaluated as being organoleptically undesirable. Thus, a tasteless and odorless sesamin extract (or purified sesamin) from sesame oil is preferably used. In addition, since sesame oil has a low sesamin content, use of sesame oil to add a desirable amount of sesamin excessively increases the volume per unit dose of a composition to be prepared. This sometimes causes inconvenience in terms of ingestion. In particular, in the case where the composition is provided as a preparation for oral administration, the preparation (tablet, capsule, etc.) becomes so bulky that it causes trouble in terms of ingestion. Thus, use of a sesamin extract (or purified sesamin) from sesame oil is preferred because the intake can be reduced. The sesamin-class compound can also be obtained by synthesis. For example, sesamin and episesamin can be synthesized by the method of Beroza et al. (J. Am. Chem. Soc., 78, 1242 (1956)).

Ergothioneine is a type of sulfur-containing amino acid. In the present invention, ergothioneine is preferably L-ergothioneine.

The salt of ergothioneine is not limited as long as it is a pharmacologically acceptable salt or a dietary acceptable salt, and it may be either an acid salt or a basic salt. Examples of the acid salt include inorganic acid salts such as hydrochloride, sulfate, nitrate, and phosphate, and organic acid salts such as acetate, citrate, maleate, malate, oxalate, lactate, succinate, fumarate, and propionate. Examples of the basic salt include alkali metal salts such as sodium salt and potassium salt, and alkaline earth metal salts such as calcium salt and magnesium salt.

Ergothioneine or a salt thereof is not limited in any way by its form, production method, or the like. Ergothioneine or a salt thereof that can be used may be a chemically synthesized product or a purified extract of a natural product. L-ergothioneine is abundantly present in golden/yellow oyster mushrooms (scientific name: Pleurotus cornucopiae var. citrinopileatus) belonging to the genus Pleurotus of the family Pleurotaceae. L-ergothioneine is also present in mushrooms such as common mushrooms (scientific name: Agaricus bisporus) including white mushrooms, cremini mushrooms, and portabella mushrooms; grey oyster mushrooms (scientific name: Pleurotus ostreatus), shiitake (scientific name: Lentinula edodes), hen-of-the-woods (scientific name: Grifola Frondosa), reishi mushrooms (scientific name: Ganoderma lucidum), lion's mane mushrooms (scientific name: Hericium erinaceus), Yanagi-matsutake (scientific name: Agrocybe aegerita), girolles (scientific name: Cantharellus cibarius), porcini mushrooms (scientific name: Boletus edulis), and common morel mushrooms (scientific name: Morchella esculenta). When L-ergothioneine is obtained from a natural product, preferably, it is extracted from a golden/yellow oyster mushroom, for example. Ergothioneine or a salt thereof can also be produced by microbial fermentation. Ergothioneine or a salt thereof may be in an isolated form.

In the composition of the present invention, the ratio of the weight of the sesamin-class compound to the weight of ergothioneine or a salt thereof in terms of ergothioneine (sesamin-class compound/ergothioneine) is 0.05 to 50. When the ratio of the weight of the sesamin-class compound to the weight of ergothioneine or a salt thereof (in terms of ergothioneine) (sesamin-class compound/ergothioneine) is in the above range, cellular senescence can be more effectively suppressed than when the sesamin-class compound or ergothioneine or a salt thereof is used alone.

Accordingly, use of the composition of the present invention even in a small amount can achieve the above effect. A smaller dose leads to easier intake, resulting in a composition that can be easily ingested continuously.

In the present invention, the ratio of the weight of the sesamin-class compound to the weight of ergothioneine or a salt thereof in terms of ergothioneine (sesamin-class compound/ergothioneine) is preferably 0.1 or more, more preferably 0.15 or more, and is preferably 20 or less, more preferably 16 or less, still more preferably 15 or less. In one embodiment, the ratio of the weight of the sesamin-class compound to the weight of ergothioneine or a salt thereof in terms of ergothioneine (sesamin-class compound/ergothioneine) is preferably 0.1 to 20, more preferably 0.15 to 16, still more preferably 0.15 to 15. A composition containing the sesamin-class compound and ergothioneine or a salt thereof in the above ratio can effectively suppress cellular senescence. Preferably, the sesamin-class compound and ergothioneine or a salt thereof are contained at the above ratio in the composition.

Cellular senescence refers to a state of irreversible arrest of cell growth. Although factors of individual senescence vary widely, it is known that aged animals show an increase in cells undergoing senescence (senescent cells) with aging and that passages of cultured primary human cells eventually result in cells that have reached their division limit and become senescent cells. Presumably, physiologically active factors secreted from senescent cells adversely affect surrounding normal cells. Senescent cells also show a decline in cellular function. Suppression of cellular senescence is effective in preventing or alleviating conditions or diseases resulting from cellular senescence or an increase in senescent cells.

Examples of the conditions or diseases resulting from cellular senescence or an increase in senescent cells include a decline in cellular function, a decline in organ function, a decline in systemic function, cancer, and chronic inflammation. Effects such as maintaining cellular function, organ function, and systemic function are expected by suppressing cellular senescence and thus suppressing an increase (accumulation) in senescent cells in subjects. An effect of preventing or alleviating conditions or diseases such as cancer and chronic inflammation is also expected.

The cellular senescence-suppressing effect of the present invention can be confirmed using a known cellular senescence marker. Examples of the cellular senescence marker include p16-INK4A (p16). p16 arrests the cell cycle in G1 phase and induces cellular senescence. Thus, increased p16 expression promotes cellular senescence. In one embodiment, preferably, the composition of the present invention is one that suppresses p16 expression. In one embodiment, the composition of the present invention can be used to suppress p16 expression.

As shown in Examples described later, use of a sesamin-class compound and ergothioneine effectively suppressed p16 expression indicating cellular senescence. The p16 expression-suppressing effect obtained by a combination of a sesamin-class compound and ergothioneine was a synergistic effect superior to an effect (additive effect) predictable from an effect obtained by use of the sesamin-class compound alone or ergothioneine or a salt thereof alone. The sesamin-class compound enhanced the p16 expression-suppressing action of ergothioneine or a salt thereof.

Thus, the combination of at least one sesamin-class compound and ergothioneine or a salt thereof can be used as active ingredients for suppressing cellular senescence. In one embodiment, the composition of the present invention can be used to suppress cellular senescence.

As shown in Examples described later, the combination of a sesamin-class compound and ergothioneine showed a replicative lifespan-extending effect on yeast cells. Extension of replicative lifespan can be regarded as extension of individual lifespan. Thus, a composition containing a sesamin-class compound and ergothioneine or a salt thereof shows an action that prevents or alleviates individual senescence (i.e., an anti-senescence action). Herein, the term “anti-senescence” refers to prevention or alleviation of individual senescence.

Sesamin-class compounds and ergothioneine or a salt thereof are present in natural products as well as food and beverages. These are compounds that have been consumed. Thus, for example, daily ingestion of sesamin-class compounds and ergothioneine or a salt thereof is less likely to cause problems in terms of safety. The present invention can provide a composition containing highly safe materials as active ingredients and having an action that suppresses cellular senescence. The composition of the present invention may be a composition for suppressing cellular senescence, which contains at least one sesamin-class compound and ergothioneine or a salt thereof as active ingredients.

Humans and other mammals usually experience a decline in metabolism and the like with aging, so that presumably, the removal rate of senescent cells slows down with aging. Presumably, suppression of cellular senescence is particularly effective in suppressing an increase in senescent cells in middle-aged and older people, for example. The term “middle-aged and older people” includes the elderly.

Suppression of cellular senescence in subjects such as humans can suppress an increase in senescent cells in subjects. In one embodiment, the composition of the present invention can be used for preventing or alleviating conditions or diseases such as a decline in cellular function, a decline in organ function, and a decline in systemic function. In one embodiment, the composition of the present invention can be used for preventing or alleviating conditions or diseases such as a decline in organ function with aging and a decline in systemic function with aging by suppressing cellular senescence. A decline in organ function with aging and a decline in systemic function with aging are examples of symptoms of individual senescence of living organisms. It has been reported that suppression of senescent cell accumulation results in an extended lifespan (Non-Patent Literature 2 described above). The composition of the present invention is also useful in preventing or alleviating individual senescence and can be used as an anti-senescence composition. The individual is preferably a human. The composition of the present invention may be an anti-senescence composition containing at least one sesamin-class compound and ergothioneine or a salt thereof as active ingredients. In one embodiment, the composition of the present invention can be suitably used as an anti-senescence composition for preventing or alleviating senescence by suppressing cellular senescence. The composition of the present invention may be an anti-senescence composition mediated by suppression of cellular senescence. A lifespan-extending effect can also be expected by suppressing cellular senescence and thus preventing or alleviating individual senescence. The composition of the present invention is also useful in extending healthy lifespan, for example.

The term “preventing” as used herein encompasses preventing onset, delaying onset, reducing the incidence rate, reducing the risk of onset, and the like. The phrase “alleviating conditions or diseases” encompasses helping a subject recover from conditions or diseases, alleviating conditions or disease symptoms, making positive changes in conditions or disease symptoms, delaying or preventing the progression of conditions or diseases, and the like.

The composition of the present invention is applicable for therapeutic use (medical use) and non-therapeutic use (non-medical use). The “non-therapeutic” is a concept that does not include medical activities, i.e., a concept that does not include methods of surgery, therapy, or diagnosis of humans.

The composition of the present invention can be provided in the form of a food or beverage, a cosmetic, a pharmaceutical product, a quasi-pharmaceutical product, feed, or the like. The composition of the present invention may be a food or beverage, a cosmetic, a pharmaceutical product, a quasi-pharmaceutical product, feed, or the like to be used for suppressing cellular senescence by itself; or the composition may be a preparation, material, or the like to be added to these products.

For example, the composition of the present invention may be provided as an agent or the like, but it is not limited thereto. The agent can be directly provided as a composition or can be provided as a composition containing the agent. In one embodiment, the composition for suppressing cellular senescence can also be referred to as a “cellular senescence suppressor”. The anti-senescence composition can also be referred to as an “anti-senescence agent”.

The composition of the present invention may be an oral composition or a parenteral composition. In one embodiment, the composition of the present invention is preferably an oral composition. The oral composition may be a food or beverage, an oral pharmaceutical product, an oral quasi-pharmaceutical product, or feed, preferably a food or beverage or an oral pharmaceutical product, more preferably a food or beverage.

In another embodiment, the composition of the present invention can be used as a topical agent for skin (topical composition for skin). The topical agent for skin may be a cosmetic, a pharmaceutical product, a quasi-pharmaceutical product, or the like, preferably a cosmetic.

The composition of the present invention can contain optional additives and optional components in addition to the at least one sesamin-class compound and ergothioneine or a salt thereof, as long as the effects of the present invention are not impaired. Such additives and components may be selected depending on the form of the composition, for example, and those generally usable in food or beverages, cosmetics, pharmaceutical products, quasi-pharmaceutical products, feed, and the like can be used. When the composition of the present invention is provided as a food or beverage, a cosmetic, a pharmaceutical product, a quasi-pharmaceutical product, feed, or the like, any common method can be used for the production. In one embodiment, the composition of the present invention may consist of at least one sesamin-class compound and ergothioneine or a salt thereof.

For example, when the composition of the present invention is provided as a food or beverage, components usable in food or beverages (e.g., food materials and optional food additives) can be added to at least one sesamin-class compound and ergothioneine or a salt thereof to provide various food or beverages. The food or beverage is not limited. Examples thereof include general food or beverages, health foods, health supplements, health drinks, foods with function claims, foods for specified health uses, and foods for the sick. The health foods, health supplements, foods with function claims, foods for specified health uses, and the like can be used in various forms of preparations such as fine granules, tablets, granules, powders, capsules, chewable tablets, dry syrups, syrups, liquid agents, beverages, energy drinks, and liquid foods.

When the composition of the present invention is provided as a cosmetic, components usable in cosmetics (e.g., cosmetically acceptable carriers and additives) can be added to at least one sesamin-class compound and ergothioneine or a salt thereof to provide various cosmetics. The cosmetics may be provided in any product form. Examples thereof include skin care cosmetics such as skin lotions, essence lotions, face packs, emulsions, creams, and sunscreen lotions.

When the composition of the present invention is provided as a pharmaceutical product or a quasi-pharmaceutical product, for example, a pharmacologically acceptable carrier, an optional additive, or the like can be added to at least one sesamin-class compound and ergothioneine or a salt thereof to provide various dosage forms of pharmaceutical products or quasi-pharmaceutical products. Such a carrier, additive, or the like may be any pharmacologically acceptable one that can be used in pharmaceutical products or quasi-pharmaceutical products.

Examples thereof include excipients, binders, disintegrants, lubricants, antioxidants, and colorants. One or more of these can be used. The form of administration (ingestion) of the pharmaceutical product or the quasi-pharmaceutical product may be oral or parenteral (e.g., transdermal, transmucosal, enteral, or injection) administration. In one embodiment, when the composition of the present invention is provided as a pharmaceutical product or a quasi-pharmaceutical product, it is preferably an oral pharmaceutical product or an oral quasi-pharmaceutical product. Examples of the dosage form for oral administration include liquids, tablets, powders, fine granules, granules, sugar-coated tablets, capsules, suspensions, emulsions, and chewable tablets.

In another embodiment, preferably, the pharmaceutical product or the quasi-pharmaceutical product is a topical agent for skin. The dosage form and the like of the topical agent for skin are not limited. The topical agent for skin can be provided in any form. Examples thereof include solutions, emulsions, creams, gels, powders, aerosols, mists, capsules, and sheets.

The pharmaceutical product may be for non-human animals.

When the composition of the present invention is provided as feed, at least one sesamin-class compound and ergothioneine or a salt thereof are simply added to feed. The feed includes feed additives. Examples of the feed include feed for farm animals such as cows, pigs, chickens, sheep, and horses; feed for small animals such as rabbits, rats, and mice; and pet food for animals such as dogs, cats, and birds.

The amount of the sesamin-class compound in the composition of the present invention is not limited and can be set according to the form of the composition or the like. The total amount of the sesamin-class compound in the composition of the present invention is, for example, preferably 0.001 wt % or more, more preferably 0.01 wt % or more, still more preferably 0.05 wt % or more, and is preferably 10 wt % or less, more preferably 5 wt % or less. In one embodiment, the total amount of the sesamin-class compound in the composition of the present invention is preferably 0.001 to 10 wt %, more preferably 0.01 to 5 wt %, still more preferably 0.05 to 5 wt %. When the composition contains two or more sesamin-class compounds, the total amount is the sum of these compounds. The amount of the sesamin-class compound can be measured by a known method such as high-performance liquid chromatography (HPLC).

The amount of ergothioneine or a salt thereof in the composition of the present invention is not limited and can be set according to the form of the composition or the like.

The amount of ergothioneine or a salt thereof in the composition of the present invention in terms of ergothioneine is, for example, preferably 0.001 wt % or more, more preferably 0.01 wt % or more, still more preferably 0.05 wt % or more, and is preferably 10 wt % or less, more preferably 5 wt % or less in the composition. In one embodiment, the amount of ergothioneine or a salt thereof in terms of ergothioneine in the composition of the present invention is preferably 0.001 to 10 wt %, more preferably 0.01 to 5 wt %, still more preferably 0.05 to 5 wt %. The amount of ergothioneine or a salt thereof can be measured by a known method such as HPLC. In the case of ergothioneine, the expression regarding the amount of ergothioneine or a salt thereof in terms of ergothioneine or an expression similar thereto refers to the amount of ergothioneine; whereas in the case of a salt of ergothioneine, the expression refers to a value obtained by multiplying the molar number of the salt by the molecular weight of ergothioneine.

The composition of the present invention can be ingested or administered using various methods known per se, depending on the form or dosage form thereof. In one embodiment, preferably, the composition of the present invention is orally ingested (orally administered). In another embodiment, when the composition of the present invention is provided as a topical agent for skin, the composition is applicable to the skin.

The administration amount (intake) of the composition of the present invention is not limited. The administration amount of the composition of the present invention may be any amount that achieves the cellular senescence-suppressing effect or anti-senescence effect, for example. The administration amount may be appropriately set according to the administration form, administration method, body weight of a subject, and the like.

In one embodiment, when the composition of the present invention is ingested by or administered to a human (adult), the total administration amount of the sesamin-class compound per day is preferably 0.5 mg or more, more preferably 1 mg or more, still more preferably 3 mg or more and is preferably 200 mg or less, more preferably 100 mg or less, still more preferably 80 mg or less. In one embodiment, in the case of a human (adult), the total administration amount of the sesamin-class compound per day is preferably 0.5 to 200 mg, more preferably 1 to 100 mg, still more preferably 3 to 80 mg. In one embodiment, preferably, sesamin and/or episesamin is orally ingested by or administered to a human (adult) in an amount of preferably 0.5 to 200 mg, more preferably 1 to 100 mg, still more preferably 3 to 80 mg per day as the total administration amount of sesamin and episesamin. When the composition contains two or more sesamin-class compounds, the total administration amount is the sum of these compounds. The above administration amount may be an administration amount per 60 kg body weight.

In one embodiment, when the composition of the present invention is ingested by or administered to a human (adult) as a subject, the administration amount of ergothioneine or a salt thereof in terms of ergothioneine per day is preferably 2 mg or more, more preferably 5 mg or more, and is preferably 50 mg or less, more preferably 25 mg or less, still more preferably 20 mg or less. In one embodiment, when the composition of the present invention is ingested by or administered to a human (adult) as a subject, the administration amount of ergothioneine or a salt thereof in terms of ergothioneine per day is preferably 2 to 50 mg, more preferably 2 to 25 mg, still more preferably 5 to 25 mg, particularly preferably 5 to 20 mg.

In one embodiment, preferably, the sesamin-class compound and ergothioneine or a salt thereof in the above amounts per day are orally ingested by or administered to a human. Preferably, the sesamin-class compound and ergothioneine or a salt thereof in the above amounts are ingested or administered in one or more portions per day, for example, in one portion or several portions (e.g., two or three portions) per day. The above administration amount may be an administration amount per 60 kg body weight.

When the composition of the present invention is used on a human in order to achieve the cellular senescence-suppressing effect or anti-senescence effect, preferably, the administration amounts of the sesamin-class compound and ergothioneine or a salt thereof are set within the above range.

In one embodiment, the composition of the present invention can be used to allow a human to ingest or be administered with the sesamin-class compound and ergothioneine or a salt thereof in the above amounts per day.

The total amount of the sesamin-class compound in the composition of the present invention per adult daily intake is preferably 0.5 to 200 mg, more preferably 1 to 100 mg, still more preferably 3 to 80 mg.

The amount of ergothioneine or a salt thereof in the composition of the present invention per adult daily intake is preferably 2 to 50 mg, more preferably 2 to 25 mg, still more preferably 5 to 25 mg, particularly preferably 5 to 20 mg, in terms of ergothioneine.

Preferably, the composition of the present invention is continuously ingested or administered. In one embodiment, preferably, the composition of the present invention is continuously ingested or administered for preferably 1 week or more, more preferably 4 weeks or more, still more preferably 8 weeks or more, particularly preferably 12 weeks or more.

The subject (administration subject) to ingest or be administered with the composition of the present invention is not limited. The administration subject is preferably a human or non-human mammal, more preferably a human.

In one embodiment, the administration subject may be, for example, a subject needing or wanting to suppress cellular senescence or a subject needing or wanting to prevent or alleviate symptoms of individual senescence.

Examples of such subject include middle-aged and older people. In one embodiment, the composition of the present invention is suitably used as a composition for suppressing cellular senescence or an anti-senescence composition for middle-aged and older people. The middle-aged and older people may be those who are 40 years old and older, for example. In one embodiment, the elderly are preferred subjects among the middle-aged and older people. The elderly may be people who are 60 years old and older or 65 years old and older, for example. For example, the composition of the present invention can be used on healthy people for purposes such as preventing conditions or diseases that are expected to be prevented or alleviated by suppressing cellular senescence.

The type of the target cells for suppression of cellular senescence is not limited. Examples thereof include fibroblasts, epithelial cells, epidermal cells, basal cells, stem cells, adipocytes, neurons, muscle cells, skeletal muscle cells, smooth muscle cells, myofibroblasts, myosatellite cells, satellite cells, osteoblasts, hepatocytes, hepatic stellate cells, kidney cells, stromal cells, blood cells, immune system cells, endothelial cells, vascular endothelial cells, pericytes, lens cells, and retinal cells. In one embodiment, the composition of the present invention is suitably used for suppressing cellular senescence of fibroblasts. Fibroblasts are essential cells for tissue support and structure formation in almost all organs.

The composition of the present invention may be labeled with one or more function claims or the like selected from the group consisting of, for example, “suppressing cellular senescence”, “suppressing conversion into senescent cells”, “preventing cellular senescence”, “preventing the senescent cell formation”, “suppressing an increase in senescent cells”, “reducing senescent cell accumulation”, “delaying cellular senescence”, “delaying progression of cellular senescence”, “suppressing cellular senescence induction”, “increasing percentage of young cells”, “extending healthy lifespan”, “healthy longevity”, “young cells”, “youthful as a whole”, and “youthful look”.

In one embodiment of the present invention, the composition of the present invention is preferably a food or beverage or a topical agent for skin labeled with one or more of these function claims, more preferably a food or beverage or a cosmetic labeled with one or more of these function claims, still more preferably a food or beverage labeled with one or more these function claims. The label may be one indicating use of the composition for obtaining the function. The label may be attached to the composition itself, or may be attached to a container or package of the composition.

The present invention also encompasses the following method:
a method of suppressing cellular senescence, the method including administering at least one sesamin-class compound and ergothioneine or a salt thereof.

Administration of at least one sesamin-class compound and ergothioneine or a salt thereof to a subject can effectively suppress cellular senescence in the subject. The anti-senescence effect can also be achieved in the subject. In the above method, preferably, the ratio of the weight of the sesamin-class compound to the weight of ergothioneine or a salt thereof in terms of ergothioneine (sesamin-class compound/ergothioneine) is 0.05 to 50.

The present invention also encompasses the following use:
use of at least one sesamin-class compound and ergothioneine or a salt thereof for suppressing cellular senescence.

In one embodiment, at least one sesamin-class compound and ergothioneine or a salt thereof can be suitably used to prevent or alleviate individual senescence by suppressing cellular senescence. In the above use, preferably, the ratio of the weight of the sesamin-class compound to the weight of ergothioneine or a salt thereof in terms of ergothioneine (sesamin-class compound/ergothioneine) is 0.05 to 50.

The above method and use may be therapeutic or non-therapeutic.

In the above method and use, the at least one sesamin-class compound, ergothioneine or a salt thereof, and preferred embodiments thereof are as described above for the composition of the present invention. Preferred ranges of the weight ratio of the at least one sesamin-class compound to ergothioneine or a salt thereof are also as described for the composition of the present invention. In the above method and use, preferably, at least one sesamin-class compound and ergothioneine or a salt thereof are administered to (ingested by) a subject at least once a day, for example, one to several times (e.g., two to three times) a day. A preferred administration method is oral administration (ingestion). The above use is preferably for humans or non-human mammals, more preferably for humans.

The above method and use only require use of at least one sesamin-class compound and ergothioneine or a salt thereof in amounts (effective amounts) that achieve a desired effect. Preferred administration amounts of the sesamin-class compound and ergothioneine or a salt thereof, preferred administration subjects, and the like are as described above for the composition of the present invention. Preferably, the sesamin-class compound and ergothioneine or a salt thereof are administered using a composition containing the same. Preferably, a composition containing at least one sesamin-class compound and ergothioneine or a salt thereof at the ratio described above (i.e., the composition of the present invention) is used.

The present invention also encompasses a method of suppressing cellular senescence, the method including administering the composition of the present invention described above. The present invention also encompasses use of the composition of the present invention for suppressing cellular senescence.

The above method and use each may include: separately preparing a sesamin-class compound (or a composition containing the same) and ergothioneine or a salt thereof (or a composition containing the same) and substantially simultaneously administering them, or administering one of them first and administering the other one later while the effect of the first one is still lasting. This achieves the effect of at least one sesamin-class compound and ergothioneine or a salt thereof (i.e., the cellular senescence-suppressing effect or anti-senescence effect) as intended by the present invention. Thus, the present invention also encompasses, for example, a kit containing a sesamin-class compound (or a composition containing the same) and ergothioneine or a salt thereof (or a composition containing the same).

The present invention also encompasses use of at least one sesamin-class compound and ergothioneine or a salt thereof for producing a composition for suppressing cellular senescence or an anti-senescence composition. The composition for suppressing cellular senescence, the anti-senescence composition, preferred embodiments thereof, and the like are as described above for the composition of the present invention.

The present invention also encompasses use of at least one sesamin-class and ergothioneine or a salt thereof for producing a composition for suppressing cellular senescence or an anti-senescence composition, wherein the ratio of the weight of the sesamin-class compound to the weight of ergothioneine or a salt thereof in terms of ergothioneine in the composition (sesamin-class compound/ergothioneine) is 0.05 to 50.

The numerical range defined by the lower limit and the upper limit herein, i.e., “the lower limit to the upper limit”, includes the lower limit and the upper limit. For example, the range defined by “1 to 2” means 1 or more and 2 or less, with 1 and 2 being inclusive. Herein, the range may be any combination of any upper limit and any lower limit.

All the academic literatures and patent literatures cited herein are incorporated herein by reference.

### EXAMPLES

The present invention is described in further detail below with reference to Examples. The present invention is not limited to these Examples.

Ergothioneine used in the following experiments is L-ergothioneine. A 1:1 mixture (by weight) of sesamin and episesamin was used as a sesamin-episesamin mixture.

### Examples 1 and 2: Test for Evaluating Action of Sesamin-Episesamin Mixture and Ergothioneine on Cellular Senescence

TIG-3 cells (human lung fibroblasts) were used to measure the expression level of p16-INK4A (p16) in the cells. Eagle's minimum essential medium supplemented with 10% fetal bovine serum was used in each of Examples 1 and 2, Reference Example 1, and Comparative Examples 1 to 4. A H₂O₂ (200 μM)-supplemented medium used for H₂O₂ treatment was prepared by adding H₂O₂ to the medium described above. The H₂O₂ (200 μM)-supplemented medium is free of sesamin-class compounds and ergothioneine.

In Examples 1 and 2, in order to investigate the action on cellular senescence, the cells were cultured in a medium containing sesamin-class compounds and ergothioneine and were then treated with the H₂O₂-supplemented medium. Subsequently, the cells were cultured in the medium containing sesamin-class compounds and ergothioneine.

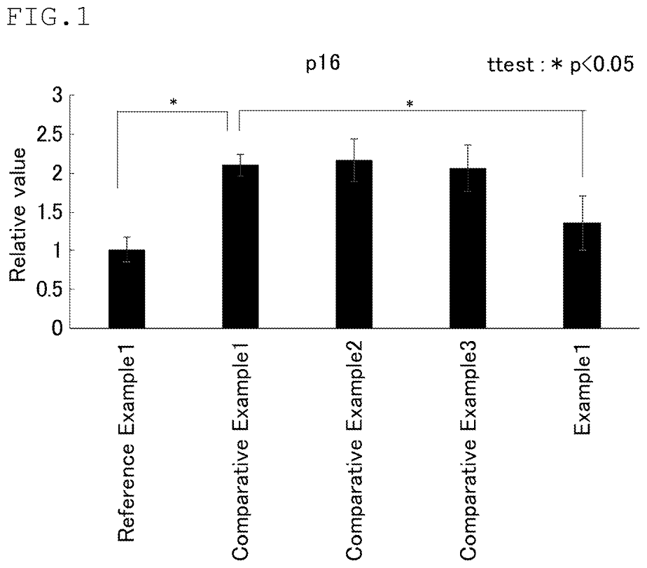

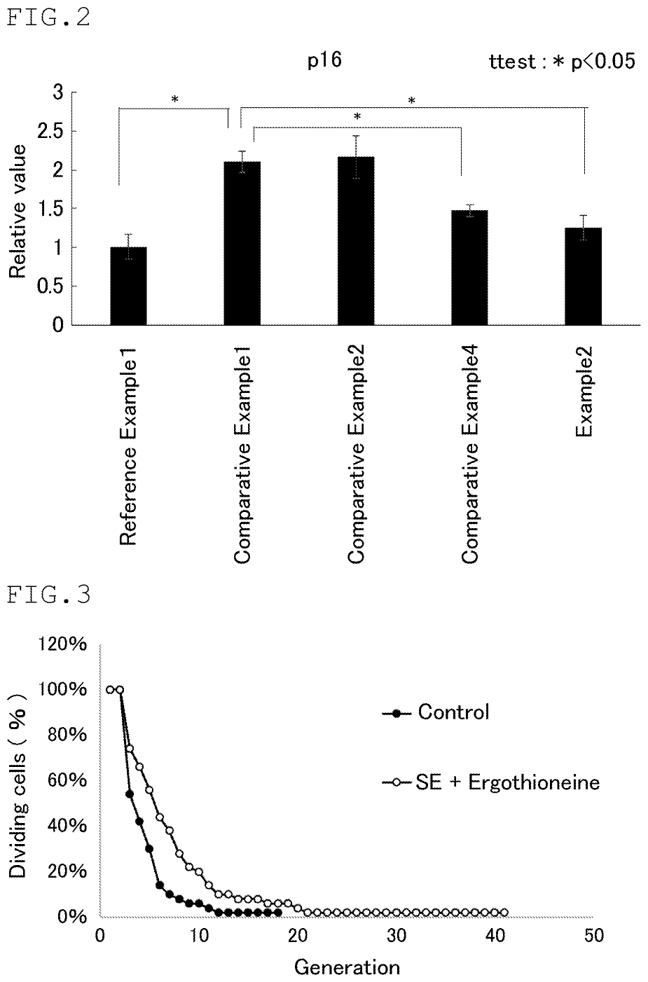

Specifically, TIG-3 cells at 4.0×10⁴ cells/well were seeded onto 6-well plates and cultured at 37° C. with CO₂ (5%) for 24 hours in a medium supplemented with a sesamin-episesamin mixture (SE) and ergothioneine at the respective concentrations shown in the following Table 1. Table 1 shows the weight ratio (SE/ergothioneine) of the sesamin-class compounds (sesamin-episesamin mixture (SE)) to ergothioneine in the medium. After 24 hours of culturing, the medium supplemented with the sesamin-episesamin mixture and ergothioneine was removed, and the cells in the wells were treated with the H₂O₂ (200 μM)-supplemented medium for two hours. Subsequently, the H₂O₂ (200 μM)-supplemented medium was again replaced with the medium supplemented with the sesamin-episesamin mixture and ergothioneine at the respective concentrations shown in Table 1, and the cells were cultured for six days. The cells cultured in the 6-well plates were collected in 500-μL QIAzol Lysis Reagent (QIAGEN). After 100 μM chloroform (Nacalai Tesque, Inc.) was added to collect the upper layer (200 μL), 200 μL isopropanol (Nacalai Tesque, Inc.) was added, followed by centrifugation (12000 g, 10 min, 4° C.). Subsequently, the supernatant was removed, and 500 μL 70% EtOH (Nacalai Tesque, Inc.) was added to pellets, followed by centrifugation (7500 g, 5 min, 4° C.). The supernatant was removed. The resulting pellets were dissolved in 20 μL DNase RNase free water (Invitrogen) and measured by NanoDrop. An RNA sample (10 μL) was mixed with an RT master mix (10 μL) (Applied Biosystems, High-Capacity cDNA Reverse Transcription Kit with RNase Inhibitor) for reaction in a thermal cycler (Applied Biosystems). The resulting synthesized cDNA (20 μL) was 1:10 diluted in DNase RNase free water, whereby a cDNA sample was prepared. A master mix (15 μL) (Takara, TB Green® Premix Ex Taq™ II (Tli RNaseH Plus)) was mixed with the cDNA sample (5 μL), and p16 was measured with a Real-Time PCR system (Applied Biosystems, StepOnePlus-01). An average p16 expression level (n=6) was determined. The results obtained are shown in FIG. 1 (Example 1) and FIG. 2 (Example 2). Significance was tested by Student's t-test (vs. H₂O₂ treatment) (*: p<0.05).

### Reference Example 1: Test for Evaluating Cellular Senescence in Untreated Group

In order to investigate the action on cellular senescence, TIG-3 cells at 1.0×10⁵ cells/well were seeded onto 6-well plates and cultured at 37° C. with CO₂ (5%) for 24 hours in a medium free of the sesamin-episesamin mixture and ergothioneine. After 24 hours of culturing, the cells were cultured for an additional two days without either treating the cells with a H₂O₂ (200 μM)-supplemented medium for two hours or replacing the medium with a medium supplemented with the sesamin-episesamin mixture and ergothioneine. In other words, in Reference Example 1, the TIG-3 cells were cultured in the medium free of the sesamin-class compound and ergothioneine for 24 hours and then cultured in the same medium for an additional two days. In Reference Example 1, the cells were not treated with the H₂O₂ (200 μM)-supplemented medium. The rest was the same as in Example 1. In this manner, p16 was measured. The results obtained are shown in FIG. 1 and FIG. 2.

### Comparative Example 1: Test for Evaluating Cellular Senescence in H₂O₂-Treated Group

In order to investigate the action on cellular senescence, TIG-3 cells at 4.0×10⁴ cells/well were seeded onto 6-well plates and cultured in a medium free of the sesamin-episesamin mixture and ergothioneine and cultured at 37° C. with CO₂ (5%) for 24 hours. After 24 hours of culturing, the cells in the wells were treated with a H₂O₂ (200 μM)-supplemented medium for two hours. Subsequently, the cells were cultured for six days without replacing the medium with a medium supplemented with the sesamin-episesamin mixture and ergothioneine. In other words, in Comparative Example 1, after treatment with the H₂O₂ (200 μM)-supplemented medium, the H₂O₂ (200 μM)-supplemented medium was replaced with a medium free of the sesamin-class compound and ergothioneine to culture the cells for an additional six days. The rest was the same as in Example 1. In this manner, p16 was measured. The results obtained are shown in FIG. 1 and FIG. 2.

### Comparative Examples 2 to 4: Test for Evaluating Cellular Senescence by Sesamin-Episesamin Mixture or Ergothioneine

In order to investigate the action on cellular senescence, TIG-3 cells at 4.0×10⁴ cells/well were seeded onto 6-well plates and cultured at 37° C. with CO₂ (5%) for 24 hours in a medium supplemented with the sesamin-episesamin mixture or ergothioneine at the respective concentrations shown in the following Table 1. After 24 hours of culturing, the medium supplemented with the sesamin-episesamin mixture or ergothioneine was removed, and the cells in the wells were treated with a H₂O₂ (200 μM)-supplemented medium for two hours. Subsequently, the H₂O₂ (200 μM)-supplemented medium was again replaced with the medium supplemented with the sesamin-episesamin mixture or ergothioneine at the respective concentrations shown in Table 1, and the cells were cultured for six days. The rest was the same as in Example 1. In this manner, p16 was measured.

The results obtained are shown in FIG. 1 (Comparative Examples 2 and 3) and FIG. 2 (Comparative Examples 2 and 4).

“H₂O₂ (UM)” in Table 1 indicates the H₂O₂ concentration in the H₂O₂ (200 μM)-supplemented medium.

TABLE 1

| | H₂O₂ | SE | Ergothioneine | SE/ergothioneine |
|---|---|---|---|---|
| | (μM) | (μM) | (μM) | (weight ratio) |
| Reference Example 1 | — | — | — | — |
| Comparative Example 1 | 200 | — | — | — |
| Comparative Example 2 | 200 | 1 | — | — |
| Comparative Example 3 | 200 | — | 0.1 | — |
| Comparative Example 4 | 200 | — | 10 | — |
| Example 1 | 200 | 1 | 0.1 | 15.5 |
| Example 2 | 200 | 1 | 10 | 0.155 |

### Results

FIG. 1 and FIG. 2 are graphs each showing results of studies on the cellular senescence-suppressing effect of the sesamin-episesamin mixture and ergothioneine. The results (relative values) shown in FIG. 1 and FIG. 2 are relative p16 expression levels in Examples and Comparative Examples, assuming the p16 expression level in Reference Example 1 as 1. Specifically, the p16 expression level (average with n=6) in each Example was divided by the p16 expression level (average with n=6) in Reference Example 1 (p16 expression level in each Example/p16 expression level in Reference Example 1) to determine a relative value. Similarly, the p16 expression level (average with n=6) in each Comparative Example was divided by the p16 expression level (average with n=6) in Reference Example 1 to determine a relative value for corresponding Comparative Example.

As shown in FIG. 1 and FIG. 2, the p16 expression level increased almost by double by the treatment with 200 UM H₂O₂ according to Comparative Example 1, compared to the control group (Reference Example 1). FIG. 1 shows no significant change in p16 expression level by the treatment with the sesamin-episesamin mixture (1 μM) or ergothioneine (0.1 μM) according to Comparative Examples 2 and 3, compared to the H₂O₂-treated group according to Comparative Example 1. In contrast, the p16 expression was significantly suppressed by the treatment with the composition containing the sesamin-episesamin mixture (1 μM) and ergothioneine (0.1 μM) (weight ratio of sesamin-class compound to ergothioneine: 15.5) according to Example 1. Next, as shown in FIG. 2, the p16 expression showed no significant change by the sesamin-episesamin mixture (1 μM) according to Comparative Example 2, compared to the H₂O₂-treated group according to Comparative Example 1, but the p16 expression was significantly suppressed by the treatment with ergothioneine (10 μM) according to Comparative Example 4. The p16 expression was significantly suppressed by the treatment with the composition containing the sesamin-episesamin mixture (1 μM) and ergothioneine (10 μM) (weight ratio of sesamin-class compound to ergothioneine: 0.155) according to Example 2. The treatment also resulted in a suppression level higher than that in Comparative Example 4 in which the cells were treated with ergothioneine alone. It was presumed from the results of Examples and Comparative Examples that the sesamin-class compounds enhanced the action of ergothioneine to suppress cellular senescence.

Based on the above, it was confirmed that the p16 expression was synergistically suppressed by the composition containing a sesamin-episesamin mixture and ergothioneine. In other words, the composition containing the sesamin-class compounds and ergothioneine synergistically shows the cellular senescence-suppressing effect, owing to the presence of both the sesamin-class compounds and ergothioneine. Thus, the composition was confirmed to exert the effect even when used in a small amount.

### Example 3: Test for Confirming Lifespan-Extending Effect of Sesamin-Episesamin Mixture and Ergothioneine

The lifespan-extending effect of the sesamin-episesamin mixture and ergothioneine was examined using yeast cells. Daughter yeast cells of K6001 yeast cells do not undergo division, so that the replicative lifespan of the parent cell can be measured by counting the number of daughter cells that bud from the parent cell. In this test, K6001 yeast cells were cultured until stationary phase in a YP galactose liquid medium (2% galactose, 1% yeast extract, 2% peptone), and a portion of the yeast cells was collected and washed twice with water. The washed yeast cells were seeded onto a YP glucose agar medium (2% galactose, 1% yeast extract, 2% peptone) containing the sesamin-episesamin mixture (10 μM) and ergothioneine (10 μM), and cultured at 30° C. for two days. Subsequently, daughter cells were counted under a microscope. The weight ratio (sesamin-episesamin mixture/ergothioneine) of the sesamin-class compound to ergothioneine in the medium was 1.55.

The control was obtained by culturing the yeast cells in the same manner as described above, except for not adding the sesamin-episesamin mixture or ergothioneine to the medium. Then, the daughter cells were counted under a microscope.

### Results

FIG. 3 is a graph showing results of studies on the lifespan-extending effect of the sesamin-episesamin mixture and ergothioneine on the yeast cells. The horizontal axis of the graph in FIG. 3 shows the generation (number) of daughter cells that bud from one parent cell. The dividing cells (%) on the vertical axis shows the percentage (%) of parent cells from which daughter cells of the generation shown on the horizontal axis budded, relative to the total number of parent cells (100%) (reference: Jarolim et al. “A novel assay for replicative lifespan in Saccharomyces cerevisiae.” FEMS yeast research 5.2 (2004): 169-177). For example, when five daughter cells (first to fifth generations) budded from a parent cell, the parent cell (dividing cell) is counted as a parent cell of each of the first to fifth generations. In FIG. 3, the black circles (•) represent the control, and the white circles (◯) represent the yeast cells cultured in the medium containing the sesamin-episesamin mixture (SE) and ergothioneine.

As shown in FIG. 3, comparison with the control shows that the treatment with the sesamin-episesamin mixture and ergothioneine increased the number of daughter yeast cells, exerting the replicative lifespan-extending effect on the yeast cells. Based on the above, it was confirmed that the composition containing both the sesamin-class compounds and ergothioneine exerts the lifespan-extending effect, owing to the presence of both the sesamin-class compounds and ergothioneine.

1. A composition comprising:
at least one sesamin-class compound; and
ergothioneine or a salt thereof,
wherein a ratio of a weight of the sesamin-class compound to a weight of ergothioneine or a salt thereof in terms of ergothioneine, i.e., sesamin-class compound/ergothioneine, is 0.05 to 50.

2. The composition according to claim 1,
wherein the sesamin-class compound is at least one of sesamin or episesamin.

3. The composition according to claim 1,
wherein the composition is an oral composition.

4. The composition according to claim 1,
wherein the composition is a food or beverage.

5. The composition according to claim 1,
wherein the composition is a cosmetic.

6. The composition according to claim 1 for use in suppressing cellular senescence.

7. The composition according to claim 1,
wherein the composition is an anti-senescence composition.

8. The composition according to claim 1,
wherein the composition is labeled with at least one selected from the group consisting of “suppressing cellular senescence”, “suppressing conversion into senescent cells”, “preventing cellular senescence”, “preventing senescent cell formation”, “suppressing an increase in senescent cells”, “reducing senescent cell accumulation”, “delaying cellular senescence”, “delaying progression of cellular senescence”, “suppressing cellular senescence induction”, “increasing percentage of young cells”, “extending healthy lifespan”, “healthy longevity”, “young cells”, “youthful as a whole”, and “youthful look”.

9. A method of suppressing cellular senescence, comprising
administering the composition according to claim 1.

10. Use of the composition according to claim 1 for suppressing cellular senescence.
